(19) United States
(12) Patent Application Publication
(10) Pub. No.: US 2020/0135327 A1
(43) Pub. Date: Apr. 30, 2020

(54) INFORMATION PROCESSING APPARATUS, INFORMATION PROCESSING METHOD, AND STORAGE MEDIUM

(71) Applicant: CANON KABUSHIKI KAISHA, Tokyo (JP)

(72) Inventors: Masahiro Yakami, Kyoto-shi (JP); Ryo Ishikawa, Kawasaki-shi (JP); Takeshi Kubo, Kyoto-shi (JP); Mizuho Nishio, Kyoto-shi (JP)

(21) Appl. No.: 16/656,090

(22) Filed: Oct. 17, 2019

(30) Foreign Application Priority Data
Oct. 26, 2018 (JP) 2018-201985

(51) Int. Cl. G16H 30/20; G16H 30/40; G06T 7/00; G06K 9/62

(52) U.S. Cl. CPC G16H 30/20; G06K 9/6202; G06T 7/0012; G16H 30/40

(57) ABSTRACT

An information processing apparatus includes an acquisition unit, a search unit, and a display control unit. The acquisition unit acquires a comparison image generated using a first examination image and a second examination image which are acquired by imaging a subject at different times/dates from each other. The search unit searches for clinical practice infonnation about a clinical practice perfonned on the subject during a period defined by a first examination date, at which the first examination image is acquired, and a second examination date, at which the second examination image is acquired. The display control unit displays the comparison image and the searched clinical practice information on a display unit.

### BACKGROUND

### Field of the Disclosure

The present disclosure relates to an information processing apparatus, an information processing method, and a storage medium.

### Description of the Related Art

In the medical field, a doctor diagnoses using medical images captured by various modalities. Especially, for a follow-up observation of the state of a subject, such as a lesion, a doctor compares a plurality of images captured at different times/dates by the same modality and observes a change in the subject made over time. Japanese Patent Application Laid-Open No. 2013-126575 discusses a technique for visualizing a change over time by presenting an image showing a difference between a reference image and a comparison image as a method for assisting a doctor in observing the change in the subject over time.

### SUMMARY

However, in a real clinical practice, a user needs to perform a clinical practice information search in order to check a change over time using a captured image and refer to associated clinical practice information, which causes inconvenience to the user.

In some embodiments, an information processing apparatus includes an acquisition unit configured to acquire a comparison image generated using a first examination image and a second examination image which are acquired by imaging a subject at different times/dates from each other, a search unit configured to search for information about a clinical practice performed on the subject during a period defined by a first examination date, at which the first examination image is acquired, and a second examination date, at which the second examination image is acquired, and a display control unit configured to display the comparison image and the searched clinical practice information on a display unit.

Various embodiments produce an advantage that a comparison of examination images captured on different examination dates and associated clinical practice information are efficiently presented to a user.

Further features of various embodiments will become apparent from the following description of exemplary embodiments with reference to the attached drawings.

### BRIEF DESCRIPTION OF THE DRAWINGS

FIG. 1 illustrates an entire configuration of a medical information processing system according to a first exemplary embodiment.

FIG. 2 is a flowchart illustrating an entire process according to the first exemplary embodiment.

FIGS. 3A and 3B are diagrams illustrating the processing in S1010 according to the first exemplary embodiment in detail.

FIG. 4 is a diagram illustrating the processing in S1050 according to the first exemplary embodiment in detail.

FIG. 5 illustrates an entire configuration of a medical information processing system according to a second exemplary embodiment.

FIGS. 6A and 6B are flowcharts illustrating an entire process according to the second exemplary embodiment.

FIG. 7 illustrates an entire configuration of a medical information processing system according to a third exemplary embodiment.

FIG. 8 is a flowchart illustrating an entire process according to the third exemplary embodiment.

### DESCRIPTION OF THE EMBODIMENTS

Various exemplary embodiments will be described in detail below with reference to the drawings. However, each component described in the exemplary embodiments is a mere example. The technical scope of various embodiments is not to be limited by the individual exemplary embodiments described below.

A medical information processing system according to a first exemplary embodiment provides the below-described functions to a user of a medical institution, such as a doctor or technologist. Specifically, the medical information processing system enables the user to observe and refer to a previous examination image of a target patient (subject of interest) to be medically treated by the user and clinical practice information. Further, if the user selects examination images captured at different times/dates as previous examination images, the medical information processing system generates and displays a comparison image suitable for comparison and observation of the plurality of examination images. Further, clinical practice information that has medically a close relationship with the comparison image is made available for reference. As used herein, the comparison image suitable for comparison and observation of the plurality of examination images refers to, for example, a temporal difference image (hereinafter, referred to as “difference image”). Further, the clinical practice information that has medically the close relationship with the comparison image refers to, for example, information about a clinical practice during a period of the examination dates of the plurality of examination images used in generating the comparison image. Specifically, the comparison image is generated and displayed to allow the user to visually recognize a change in the patient on the examination images during the examination period and also allow the user to refer to clinical practice information about a highly-relevant clinical practice that can have a medical cause-and-effect relationship with the change. This increases a working efficiency for the user.

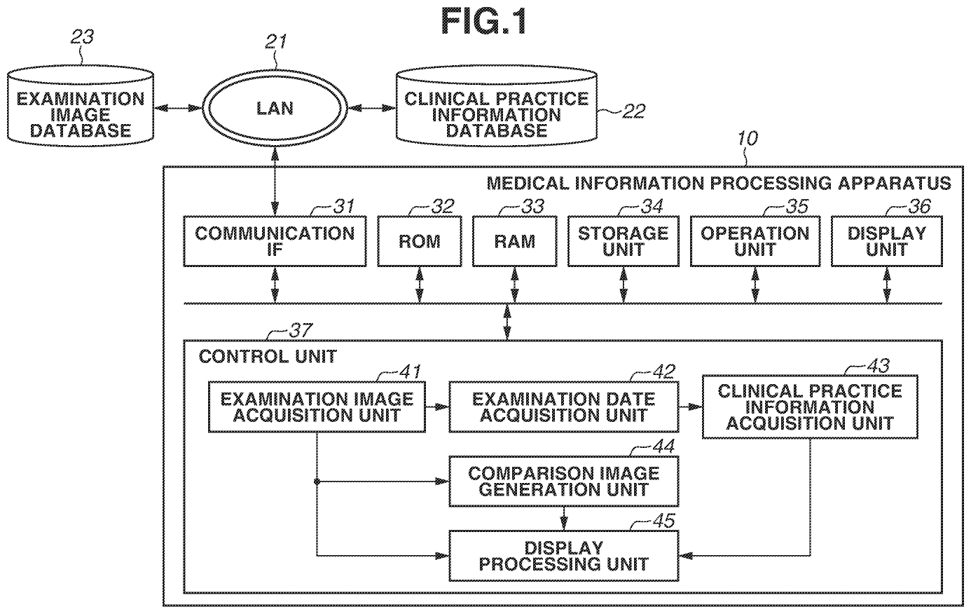

FIG. 1 illustrates an entire configuration of the medical information processing system according to the first exemplary embodiment. The medical information processing system includes a medical information processing apparatus 10 (information processing apparatus), a clinical practice information database 22, and an examination image database 23, which are communicably connected to one another via a communication unit. While the communication unit is a local area network (LAN) 21 in the present exemplary embodiment, the communication unit can be a wide area network (WAN). Further, a connection method can be either a wired connection method or a wireless connection method.

The clinical practice information database 22 stores a plurality of pieces of previous clinical practice information about a plurality of patients. Each piece of clinical practice information contains a patient name (patient identifier (ID)), clinical practice date (or clinical practice time/date), and clinical practice details (examination name, examination result, diagnosis name, radiogram interpretation report information, medication information, medical treatment information, chief complaint of the patient). The above-listed types of information contained in the clinical practice information are mere examples and are not intended to limit the content of the clinical practice information. Further, each piece of clinical practice information is provided with a unique number (clinical practice information ID) by which the clinical practice information can be discriminated from the other clinical practice information, and the medical information processing apparatus 10 reads information based on the unique number. Besides the information reading function, the clinical practice information database 22 also provides functions, such as a list display function, a summary display function, a search function, and a writing function, in cooperation with the medical information processing apparatus 10.

The examination image database 23 stores examination images of a plurality of patients and supplementary information about the examination images. The term “examination image” refers to, for example, a medical image captured with a diagnostic imaging apparatus (modality), such as a computer tomographic (CT) or magnetic resonance imaging (MRI) apparatus, and may be various images, such as a two-dimensional image, three-dimensional image, monochrome image, and color image. Further, the term “supplementary information about an examination image” refers to a patient name (patient ID), examination date information (date of capture of the examination image), and name of the modality by which the examination image has been captured. The examination date information may contain not only the date of capture of the examination image but also the time of capture of the examination image. In other words, the examination date information may be examination date and time. Specific examples of the supplementary information include, but are not limited to, a Digital Imaging and Communication in Medicine (DICOM) tag in the examination image as defined by the DICOM standard. The supplementary information may be any information that is identifiable based on the examination image. Further, the above-described information contained in the supplementary information are mere examples and the content of the supplementary information is not limited to those described above. Further, a unique number (examination image ID) is provided to each examination image and supplementary information about the examination image, so that the examination image and the supplementary information can be discriminated from other information, and the medical information processing apparatus 10 reads information based on the unique number. In a case of an examination image that is a three-dimensional volume image formed by a plurality of two-dimensional tomographic images, the examination image ID is provided to each of the two-dimensional tomographic images and the three-dimensional volume image which is the set of the two-dimensional tomographic images. Besides the information reading function, the examination image database 23 also provides other functions, such as an examination image list display function, a thumbnail display function, a search function, and an information writing function, in cooperation with the medical information processing apparatus 10.

The medical information processing apparatus 10 acquires information stored in the clinical practice information database 22 or the examination image database 23 via the LAN 21. A functional configuration of the medical information processing apparatus 10 includes a communication interface (IF) 31, a read-only memory (ROM) 32, a random access memory (RAM) 33, a storage unit 34, an operation unit 35, a display unit 36, and a control unit 37.

The communication IF 31 is realized by, for example, a LAN card and performs communication between an external apparatus (e.g., the clinical practice information database 22 and the examination image database 23) and the medical information processing apparatus 10 via the LAN 21. The ROM 32 is realized by a non-volatile memory and stores various programs. The RAM 33 is realized by a non-volatile memory and temporarily stores various types of information. The storage unit 34 is realized by, for example, a hard disk drive (HDD) and stores various types of information. The operation unit 35 is realized by, for example, a keyboard and a mouse and inputs a user instruction into the medical information processing apparatus 10. The display unit 36 is realized by, for example, a display and displays various types of information to a user (e.g., doctor). The operation unit 35 and the display unit 36 are controlled by the control unit 37 to provide a function as a graphical user interface (GUI).

The control unit 37 is realized by, for example, a central processing unit (CPU) and comprehensively controls processing performed in the medical information processing apparatus 10. A functional configuration of the control unit 37 includes an examination image acquisition unit 41, an examination date acquisition unit 42, a clinical practice information acquisition unit 43, a comparison image generation unit 44, and a display processing unit 45.

The examination image acquisition unit 41 acquires an examination image of a patient from the examination image database 23 via the communication IF 31 and the LAN 21 according to a user operation input via the operation unit 35. The examination image acquisition unit 41 outputs the acquired examination image to the examination date acquisition unit 42 and the comparison image generation unit 44.

The examination date acquisition unit 42 acquires examination date information about the examination image acquired by the examination image acquisition unit 41. The examination date acquisition unit 42 outputs the acquired examination date information to the clinical practice information acquisition unit 43.

The clinical practice information acquisition unit 43 performs the processing, which will be described below, based on the examination date information acquired by the examination date acquisition unit 42 and reads clinical practice information to be presented to the user from the clinical practice information database 22 via the communication IF 31 and the LAN 21. The clinical practice information acquisition unit 43 outputs the read clinical practice information to the display processing unit 45.

The comparison image generation unit 44 performs the processing, which will be described below, based on the examination image acquired by the examination image acquisition unit 41 and generates a comparison image. Alternatively, the comparison image generation unit 44 may acquire a pre-generated comparison image. The comparison image generation unit 44 outputs the generated comparison image to the display processing unit 45.

The display processing unit 45 displays, on the display unit 36, the comparison image generated by the comparison image generation unit 44 and the clinical practice information acquired by the clinical practice information acquisition unit 43.

At least a portion of the components of the control unit 37 can be realized as an independent apparatus. Further, each component can be realized as software that realizes a function. In this case, the software that realizes a function may operate on a server via a network, such as the cloud. In the present exemplary embodiment, each component is realized by software in a local environment.

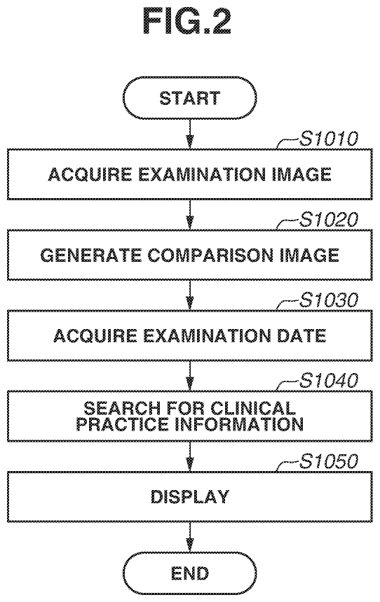

Next, an entire process that is performed by the medical information processing apparatus 10 according to the present exemplary embodiment will be described in detail below with reference to FIG. 2. Further, while a case of using a CT image as an examination image will be described as an example below, the implementation of the present exemplary embodiment is not limited to that described below.

### (S1010) <Acquisition of Examination Image>

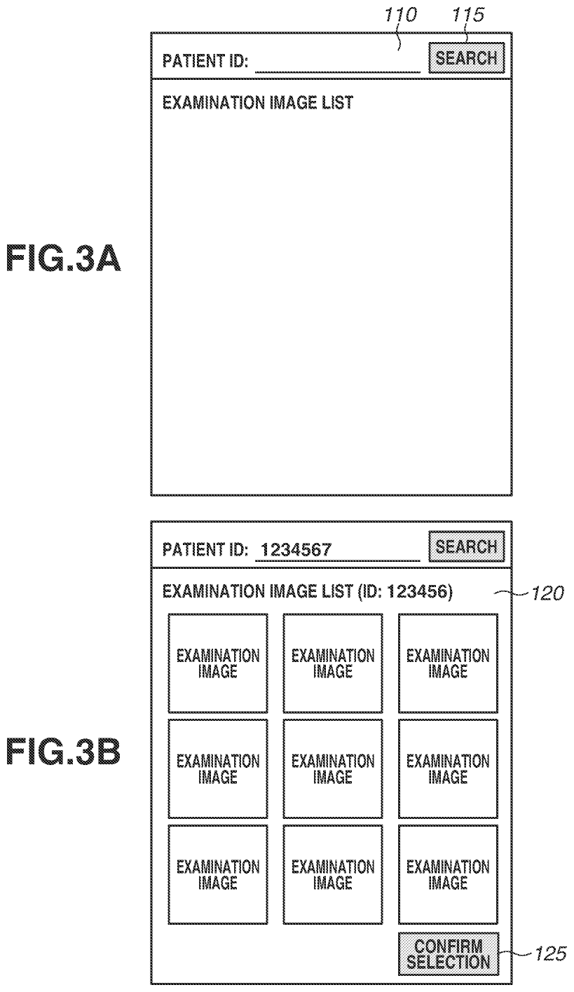

In S1010, the examination image acquisition unit 41 performs processing to acquire a processing target examination image from the examination image database 23. The processing is perfoimed when a user operation is received via the GUI provided by the operation unit 35 or the display unit 36. This is realized by, for example, displaying a screen as illustrated in FIG. 3A on the display unit 36 and providing the GUI configured to receive user input via the operation unit 35. At this time, if the user inputs the patient ID of an observation target patient to a patient ID input section 110 and then presses a search button 115 in FIG. 3A, the control unit 37 transmits and receives information to and from the examination image database 23 via the examination image acquisition unit 41. Then, the control unit 37 searches the examination image database 23 for an examination image of the patient and presents the result of the search to the user by displaying an examination image thumbnail list 120 as illustrated in FIG. 3B. At this time, the thumbnail list 120 displays a thumbnail list of examination images acquired in a plurality of previous examinations on the patient. At this time, each thumbnail may be displayed adding the supplementary information about the examination image. For example, the examination date of each examination image may be displayed near the thumbnail image or superimposed on the thumbnail image.

The case of searching for the observation target patient using the patient ID has been described above, as an example. However, the implementation of the present exemplary embodiment is not limited to the above-described case, and the search may be performed using a patient name. In this case, one of the first name and the last name of the observation target patient may be used as a condition in the search. As a result of the search, if a plurality of patients matches the name, a list of the plurality of matched patients may be displayed on a popup screen to provide a GUI via which an observation target patient is selectable from the list displayed on the popup screen.

The GUI can also receive desired two examination images (first and second examination images) selected from the thumbnail list 120 by a user operation. Specifically, each thumbnail image on the thumbnail list 120 can be switched between a selected state and an unselected state by an operation of the mouse on the operation unit 35. Then, a first examination image and a second examination image of the patient selected as an observation target by the user are acquired based on the selected/unselected state of the thumbnail images at the time when a “CONFIRM SELECTION” button 125 is clicked with the mouse. At this time, if the user selection does not satisfy a predetermined condition, a warning is displayed, and a display to prompt the user to re-select an examination image is produced. The predetermined condition refers to, for example, a case in which the number of selected images is not two or a case in which the two selected images are different in the type of the image capturing apparatus. Other conditions can also be used, such as a condition that the examination dates of the two selected images are not the same or a condition that the interval between the examination dates is within a predetermined range. Further, in a case in which the user selects one examination image from the thumbnail list 120, only an examination image which satisfies the above condition may be set to be additionally selectable. Further, an examination image which is not likely to satisfy the condition of any other image as a set may be not displayed on the thumbnail list 120.

Further, the implementation of some embodiments is not limited to the above-described example. For example, a list of examination dates of examination images of an observation target patient may be displayed, and a reception unit configured to receive input regarding an examination date may be provided so that the user can designate an examination date (first and second examination dates) of a processing target examination image from the list of examination dates. In this case, an examination image of the received examination date is determined as a processing target. Then, in S1020 described below, a comparison image generated using the first examination image specified based on the first examination date and the second examination image specified based on the second examination date is acquired. Further, the processing of acquiring an examination date of an examination image in S1030 described below can be omitted, and S1040 and the subsequent are performed based on the user-designated examination date received by the above-described processing.

The examination image acquisition unit 41 performs the above-described processing to thereby read the information (image data, supplementary infoiiiiation) about the first and second examination images from the examination image database 23, and the medical information processing apparatus 10 stores the read information using the ROM 32, the RAM 33, or the storage unit 34.

### (S1020) <Generation of Comparison Image>

In S1020, the comparison image generation unit 44 performs processing to generate a comparison image for comparing the first and second examination images acquired in S1010. Specifically, the comparison image generation unit 44 corresponds to an example of an acquisition unit configured to acquire a comparison image generated using a first examination image and a second examination image which are acquired by imaging a subject at different times/dates from each other. As an example of a method of generating a comparison image, a difference image that specifies a difference between the first and second examination images is generated as a comparison image. In this case, it is desirable to perform difference processing after a difference in image quality between the images or a difference (defoimation) in position/shape of the subject in the first and second examination images is accommodated. The correction of a deformation between the images, and the accommodation of a difference in image quality may be performed using any publicly-known method, and detailed description thereof is omitted.

The method of generating a comparison image is not limited to the above-described method and, for example, a superimposed image generated by superimposing the first and second examination images on top of another may be generated as a comparison image. In this case, the superimposed image may be generated by assigning different color channels to the first and second examination images and mixing them together to generate a superimposed image as a color image. Further, the method of generating a comparison image is not limited to the above-described method and, for example, an image in which the first and second examination images are arranged next to each other may be generated as a comparison image.

The comparison image generation unit 44 performs the above-described processing to generate a comparison image, and the medical information processing apparatus 10 stores the generated comparison image. While the processing of generating a comparison image after receiving the user selection of an examination image in S1010 is described in the present exemplary embodiment, if a comparison image has been generated in advance, the generated comparison image may be acquired without generating a new comparison image.

### (S1030) <Acquisition of Examination Date>

In S1030, the examination date acquisition unit 42 performs processing to acquire the examination dates of the first and second examination images acquired in S1010. As described above, the examination dates (image capturing dates) of the first and second examination images are recorded as the supplementary information, and in S1030, the examination dates of the first and second examination images are acquired from the supplementary information. While the examination dates are used in the present exemplary embodiment, examination times and dates or image capturing times and dates may be used. The examination date acquired from the first examination image will be referred to as “first examination date”, whereas the examination date acquired from the second examination image will be referred to as “second examination date”.

In the present exemplary embodiment, a case in which the first examination date is Apr. 25, 2010 and the second examination date is Aug. 21, 2014 will be described below as an example.

### (S1040) <Search for Clinical Practice Information>

In S1040, the clinical practice information acquisition unit 43 perform processing to acquire, from the clinical practice information database 22, clinical practice information suitable for reference together with the comparison image based on the first and second examination dates acquired in S1030. Specifically, the processing is performed by the below-described procedure.

First, the clinical practice information acquisition unit 43 acquires, by searching only the clinical practice information about the same patient as the target patient of the examination image acquired in S1010 from the plurality of pieces of clinical practice information recorded in the clinical practice information database 22. This processing is performed by referring to the patient ID contained in the supplementary information of the examination image acquired in S1010 and searching for the clinical practice information having the same patient ID.

Next, the clinical practice information acquisition unit 43 extracts, from the search result, the clinical practice infon iation about a clinical practice perfoiined during a period between the first and second examination dates, based on the first and second examination dates acquired in S1030. Specifically, the clinical practice information acquisition unit 43 corresponds to an example of a search unit configured to search for information about a clinical practice performed on the subject during a period defined by a first examination date, at which the first examination image is acquired, and a second examination date, at which the second examination image is acquired. This processing is performed by calculating whether a clinical practice date which is supplementary information about the clinical practice information is before or after the first or second examination date. At this time, the clinical practice information of the same date as the first examination date and the clinical practice information of the same date as the second examination date may be included in, or excluded from the search result. In other words, the period may be a time between the first examination date and the second examination date including the first examination date and the second examination date or a time between the first examination date and the second examination date excluding the first examination date and the second examination date.

The clinical practice information acquisition unit 43 records the clinical practice information that is eventually extracted by the above-described procedure. Specifically, the clinical practice information acquisition unit 43 records the clinical practice information ID of the extracted clinical practice information. In a case in which a plurality of pieces of clinical practice information is extracted, the clinical practice information ID of each piece of extracted clinical practice information is recorded.

While the case in which clinical practice information to be a target is extracted from the plurality of pieces of clinical practice information recorded in the clinical practice information database 22 based on the patient ID and the clinical practice date is described above as an example, the implementation of some embodiments is not limited to that described above. For example, only the clinical practice information that describes a treatment detail that is more likely to cause a change in an examination image may be extracted. For example, clinical practice information that describes that a cancer patient is treated with a drug having an effect of reducing a focus of disease is medically highly relevant to a change in an examination image of the patient. Further, clinical practice information that describes the patient who is surgically treated (surgical treatment) or is treated by radiation is highly relevant to a change in an examination image of the patient. Only the clinical practice information that is highly relevant to a change in the examination image as described above may be extracted. For example, a region of the patient body where a difference is significantly exhibited in a comparison image is detected, and clinical practice information containing information about a treatment of the detected region is extracted. That is, this processing corresponds to an example of the search unit that searches, of the clinical practice information based on the period for clinical practice information, for information that specifies a detail of a clinical practice that could be a possible cause of a change between the first examination image and the second examination image.

In the present exemplary embodiment, an example of a case in which clinical practice information about four days that are Oct. 20, 2010, Jul. 15, 2011, Mar. 8, 2012, and Dec. 20, 2013 are extracted by the search in S1040 as clinical practice information within the period between the first examination date (Apr. 25, 2010) and the second examination date (Aug. 21, 2014).

### (S1050) <Display>

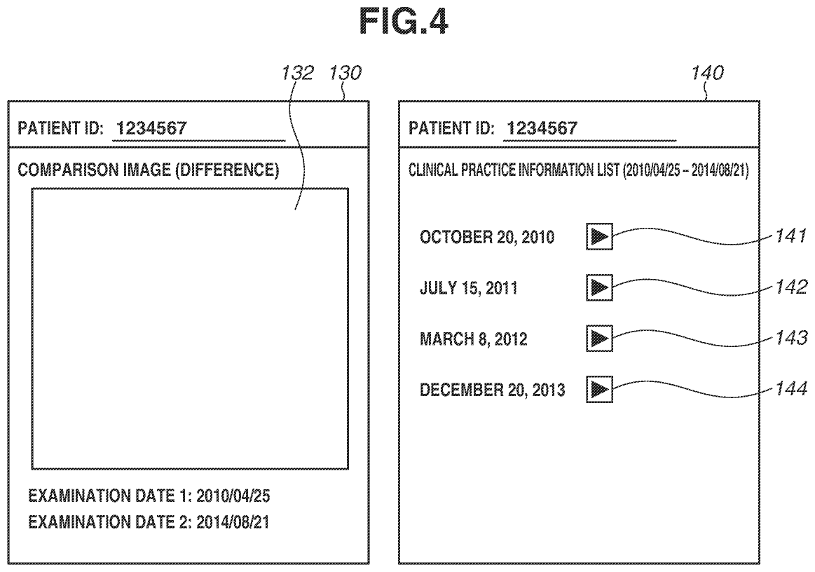

In S1050, the display processing unit 45 displays, on the display unit 36, the comparison image generated in S1020 and the clinical practice information acquired in S1040. In other words, the display processing unit 45 corresponds to an example of a display control unit configured to display the comparison image and the extracted clinical practice information on a display unit. FIG. 4 illustrates a specific example of a method of displaying on the display unit 36. In FIG. 4, displaying of the comparison image and the clinical practice information next to each other is illustrated as an example. A comparison image display window 130 in FIG. 4 is a window that displays the comparison image generated in S1020 and associated information. An image window 132 is provided within the comparison image display window 130 and displays the comparison image. In the case where the comparison image is a three-dimensional image, sliced images of the comparison image are displayed in the image window 132 in such a manner that the sliced images can be switched by a user operation received from the operation unit 35. Further, a clinical practice information display window 140 is provided on the right hand side of the comparison image display window 130 and displays the clinical practice information acquired in S1040. In the present exemplary embodiment, a list of clinical practice dates of the clinical practice information extracted by the search in S1040 is displayed. On the right hand sides of the respective clinical practice dates, reference buttons 141 to 144 for referring to the clinical practice information are displayed. If the user presses the reference button 141, 142, 143, or 144 displayed on the right hand side of the clinical practice information that the user desires to refer to, another screen is laid out and the clinical practice information is displayed on the laid-out screen so that the user can refer to the displayed clinical practice information. For example, in a case where the user desires to refer to the clinical practice information about Jul. 15, 2011 from the clinical practice information list, if the user presses the reference button 142 on the right hand side of the clinical practice date, the clinical practice information is displayed so that the user can refer to the clinical practice information. While the example of displaying the comparison image display window 130 and the clinical practice information display window 140 is described above, the comparison image and the clinical practice information may be displayed in the same window. Further, the positional relationship between the comparison image display window 130 and the clinical practice information display window 140 is not limited to that illustrated in FIG. 4. The clinical practice information display window 140 may be displayed on the left hand side, upper side, or lower side of the comparison image display window 130. Just the comparison image and the clinical practice information are to be displayed in such a manner that the user can compare the comparison image and the clinical practice information.

The above-described technique makes it possible for a user to observe and compare the first examination image and the second examination image which are acquired by imaging the subject at different times/dates and also makes it possible to provide the user with clinical practice information relevant to the comparison image. Further, the above-described technique solves a problem of the processing load on various apparatuses caused by conducting an unnecessary search due to erroneous input in a manual search for the clinical practice information relevant to the comparison image. This is another advantage.

### MODIFIED EXAMPLE 1-1

While the comparison image and the clinical practice information are displayed in S1050 and then the process is ended as an example of the present exemplary embodiment, the implementation of some embodiments is not limited to that described above. For example, following S1050, the medical information processing apparatus 10 may receive and record a result of comparison radiogram interpretation performed by the user based on the comparison image and the clinical practice information. Specifically, the user can add an annotation to an interesting region for the user—(e.g., region where a difference between examination images is recognized on the comparison image) on the comparison image displayed on the image window 132 in FIG. 4. Further, clinical findings regarding the region can be recorded in text format in association with the annotation. At that time, the user can select, from the clinical practice information displayed in S1050, information that is significantly relevant to the difference between the examination images, and the user can add a link to the selected clinical practice information as information associated with the clinical findings. New clinical practice information containing a link to both the clinical findings about the comparison image and the clinical practice information may be generated. The above-described technique makes it possible to record the recognized difference between the examination images on the comparison image in association with the clinical practice information about a previous clinical practice that causes the difference. Thus, for example, the previous drug administration to the patient can be recorded in association with cure observation of the disease shown on the examination images, and the user can refer to the information again later. Specifically, the above-described processing corresponds to an example of a recording unit configured to record the comparison image and the extracted clinical practice information in association with each other.

The above-described method produces an advantage that a result of radiogram interpretation performed by the user based on the displayed comparison image and the displayed clinical practice information is efficiently recorded.

### MODIFIED EXAMPLE 1-2

While the case of displaying the comparison image in S1050 together with the clinical practice information acquired by the search in S1040 is described as an example in the present exemplary embodiment, the implementation of some embodiments is not limited to that described above. For example, the clinical practice information to be displayed in S1050 may be the clinical practice information about all the previous clinical practices performed on the patient. In that case, the clinical practice information acquired by the search in S1040 may be displayed in such a manner that the clinical practice information is discriminable from the other clinical practice information.

Specifically, in S1020, the comparison image generation unit 44 acquires the comparison image, and in S1040, all the pieces of clinical practice information about the previous clinical practices performed on the patient are acquired. Then, in the case of displaying the acquired comparison image and the acquired clinical practice information on the display unit 36, whether the clinical practice date of each piece of the clinical practice information is within the period from the first examination date to the second examination date is determined. The clinical practice information about a clinical practice that is performed during the period is displayed using red characters, whereas the rest is displayed using black characters. The display form of the clinical practice information is not limited to that described above. The thickness of the characters may be changed, and/or the color of a frame line surrounding the clinical practice information may be changed. Further, an icon or image may be displayed near the clinical practice information. Just the clinical practice information about the clinical practice that has been performed during the period, is to be displayed in such a manner that the clinical practice information is discriminable from the other clinical practice information.

Specifically, Modified Example 1-2 corresponds to an information processing apparatus including an acquisition unit configured to acquire a comparison image generated using a first examination image and a second examination image which are acquired by imaging a subject at different times/dates from each other and clinical practice information about a clinical practice performed on the subject, a display control unit configured to display the comparison image and the clinical practice information on a display unit, and a determination unit configured to determine whether the clinical practice relating to the clinical practice information has been performed during a period defined by a first examination date, at which the first examination image is acquired, and a second examination date, at which the second examination image is acquired. The display control unit displays the clinical practice information on the display unit in such a manner that the clinical practice information is discriminable from other clinical practice information, based on a result of the determination.

The above-described technique produces an advantage that the user can refer to all the pieces of clinical practice information about previous clinical practices performed on the patient and can also recognize the clinical practice information that is highly relevant to the comparison image.

### MODIFIED EXAMPLE 1-3

While the case of displaying the comparison image in S1050 together with the clinical practice information has been described as an example in the present exemplary embodiment, the implementation of some embodiments is not limited to that described above. For example, only the clinical practice information may be displayed without displaying the comparison image. In this case, the processing of generating the comparison image in S1020 may be omitted. The above-described technique provides a system by which the user selects examination images of different examination dates and can refer to the clinical practice information that is relevant to the examination period between the selected examination images. Further, a system may be provided in which the user can set whether to generate and display a comparison image, and the processing may be switched according to the user setting.

### MODIFIED EXAMPLE 1-4

While the example of performing a search for clinical practice information about a clinical practice that is performed during the period from the first examination date to the second examination date in S1040 has been described in the present exemplary embodiment, the implementation of some embodiments is not limited to that described above. For example, in a case in which a medication or medical treatment is performed on the patient before the first examination date, an effect of the medication or medical treatment may be presented during the period. In such a case, it is desirable to present, to the user, not only the details of the clinical practices that are performed during the period but also the details of some of the clinical practices that are performed before the period.

Thus, in S1040, the clinical practice information acquisition unit 43 subtracts a predetermined number of dates from the first examination date and calculates a date before the first examination date. Then, the clinical practice information acquisition unit 43 searches for clinical practice information about a clinical practice performed during a period from the calculated date to the second examination date. The predetermined number of dates to be used can be a number of dates that is stored in advance in the storage unit 34 or a number of dates that is input by the user.

Furthermore, for example, the clinical practice information acquisition unit 43 may check whether there is clinical practice information about a clinical practice that is performed before the first examination date and may have affected the patient during the period between the first examination date and the second examination date. Then, if there is such clinical practice information, a search for clinical practice information during a period between the date at which the clinical practice is performed and the second examination date may be performed.

The above-described technique makes it possible to present also the details of part of the clinical practices that are perfonned before the period.

In the first exemplary embodiment, an example of the medical information processing apparatus configured to receive selection of the first and second examination images from the user has been described. In a second exemplary embodiment, a medical information processing apparatus configured to receive selection of a comparison image from a user and search for clinical practice information to be associated with the selected comparison image based on a period from an examination date of a first examination image used in generating the selected comparison image to an examination date of a second examination image will be described below.

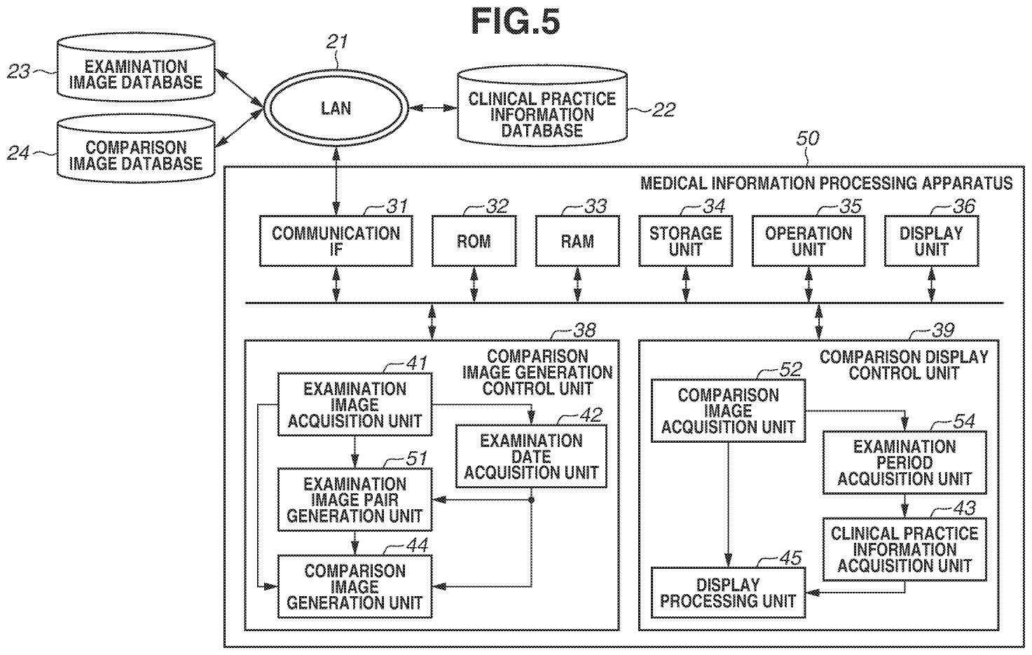

FIG. 5 illustrates an entire configuration of the medical information processing system according to the second exemplary embodiment. The medical information processing system includes a medical information processing apparatus 50, the clinical practice information database 22, the examination image database 23, and a comparison image database 24, all of which are communicably connected to one another via the communication unit. A function similar to that of the configuration described in the first exemplary embodiment is given the same reference numeral, and detailed description thereof is omitted.

The comparison image database 24 is a database configured to record a comparison image and supplementary information about the comparison image generated by the medical information processing apparatus 50, which will be described below. A plurality of pieces of information is recorded in the comparison image database 24, and a unique number (comparison image ID) is provided to each piece of the stored information. With respect to the comparison image database 24, a function is provided such as an information reading function, an information writing function, a list display function, a thumbnail image display function, and a search function, in cooperation with the medical information processing apparatus 50.

The medical information processing apparatus 50 acquires, via the LAN 21, information stored in the clinical practice information database 22, the examination image database 23, or the comparison image database 24. A functional configuration of the medical information processing apparatus 50 includes the communication IF 31, the ROM 32, the RAM 33, the storage unit 34, the operation unit 35, the display unit 36, a comparison image generation control unit 38, and a comparison display control unit 39.

The comparison image generation control unit 38 is realized by, for example, a CPU and comprehensively controls the comparison image generation processing performed by the medical information processing apparatus 50. Details of the comparison image generation processing will be described below. A functional configuration of the comparison image generation control unit 38 includes the examination image acquisition unit 41, the comparison image generation unit 44, the examination date acquisition unit 42, and an examination image pair generation unit 51.

The examination image pair generation unit 51 generates a pair of examination images from which the comparison image generation unit 44 generates a comparison image, which will be described below, based on the examination images acquired by the examination image acquisition unit 41 and the examination dates acquired by the examination date acquisition unit 42. Then, the examination image pair generation unit 51 outputs information about the generated pair of examination images to the comparison image generation unit 44.

The comparison display control unit 39 is realized by, for example, a CPU and comprehensively controls the comparison display processing performed by the medical information processing apparatus 50. Details of the comparison display processing will be described below. A functional configuration of the comparison display control unit 39 includes a comparison image acquisition unit 52, an examination period acquisition unit 54, the clinical practice information acquisition unit 43, and the display processing unit 45.

The comparison image acquisition unit 52 acquires, according to a user operation input via the operation unit 35, the comparison image of the patient and the supplementary information about the comparison image from the comparison image database 24 via the communication IF 31 and the LAN 21. The comparison image acquisition unit 52 outputs the acquired comparison image and the acquired supplementary information about the comparison image to the examination period acquisition unit 54 and the display processing unit 45.

The examination period acquisition unit 54 acquires examination date information from the supplementary information about the comparison image acquired by the comparison image acquisition unit 52. Then, the examination period acquisition unit 54 outputs the acquired examination date information to the clinical practice information acquisition unit 43.

At least one or some of the components of the comparison image generation control unit 38 and the comparison display control unit 39 may be realized as a separate apparatus. Further, a single control unit may include the components of the comparison image generation control unit 38 and the comparison display control unit 39. Further, the components may be realized as software configured to realize the functions. In this case, the software configured to realize the functions may be operated on a server via a network such as the cloud. In the present exemplary embodiment, the components are each realized by software in a local environment.

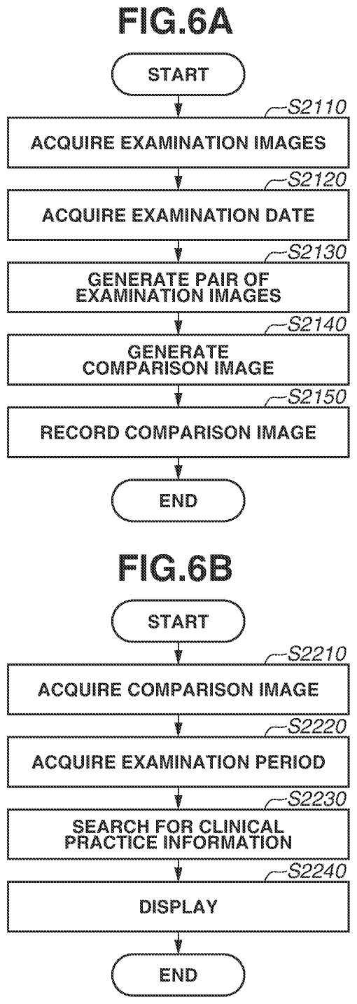

Next, an entire procedure of the processing performed by the medical information processing apparatus 50 according to the present exemplary embodiment will be described in detail below with reference to FIGS. 6A and 6B. The medical information processing apparatus 50 according to the present exemplary embodiment performs two types of processing, i.e., the comparison image generation processing and the comparison display processing. The comparison image generation processing is the processing of generating a plurality of comparison images based on examination images recorded in the examination image database 23. The comparison display processing is the processing of displaying the comparison images generated by the comparison image generation processing and clinical practice information relevant to the generated comparison images in such a manner that the user can observe and refer to the comparison images and the clinical practice information relevant to the comparison images. The comparison image generation processing is performed prior to the comparison display processing.

FIG. 6A is a flowchart illustrating the comparison image generation processing performed by the medical information processing apparatus 50. A procedure of the processing performed as the comparison image generation processing by the medical information processing apparatus 50 will be described in detail below according to an order specified in the flowchart.

### (S2110) <Acquisition of Examination Images>

In S2110, the comparison image generation control unit 38 controls the examination image acquisition unit 41 and acquires an examination image of the subject of interest from the examination images recorded in the examination image database 23. In the present exemplary embodiment, a case in which the examination image database 23 records a plurality of examination images of the subject of interest on a plurality of examination dates will be described as an example. In S2110, all the examination images are acquired.

### (S2120) <Acquisition of Examination Date>

In S2120 the comparison image generation control unit 38 controls the examination date acquisition unit 42 and performs processing to acquire the examination date of each of the plurality of examination images acquired in S2110. The processing performed to acquire the examination date of each of the plurality of examination images is similar to that in S1030 in the first exemplary embodiment. The processing is performed on each of the plurality of examination images acquired in S2110, and the examination date of each of the examination images is acquired.

### (S2130) <Generation of Pair of Examination Images>

In S2130, the comparison image generation control unit 38 controls the examination image pair generation unit 51 and performs processing to generate a pair of examination images for use in the below-described processing of generating a comparison image. A specific example of the processing will be described below. First, the examination image pair generation unit 51 generates every unique combination of all the examination images acquired in S2110 as candidate pairs of examination images. Further, a pair that matches a plurality of conditions among the candidate pairs is generated as a pair of examination images. Examples of the conditions include a condition that the examination images of the pair have been captured by the same modality, a condition that the acquired examination dates of the examination images of the pair are different from each other, and a condition that an interval between the acquired examination dates is within a predetermined range (e.g., one month or longer and three years or shorter). The pair of examination images is generated by the above-described processing.

### (S2140) <Generation of Comparison Image>

In S2140, the comparison image generation control unit 38 controls the comparison image generation unit 44 and performs processing to generate a comparison image of each pair of examination images generated in S2130. In this processing, the processing similar to the processing of S1010 and S1020 in the first exemplary embodiment is perfoiiiied on each pair of examination images. Detailed description thereof is omitted herein. Further, the comparison image generation unit 44 records, in the supplementary information about the generated comparison image, examination date information about each examination image used in generating the comparison image.

### (S2150) <Recording of Comparison Image>

In S2150, the comparison image generation control unit 38 records, in the comparison image database 24, information about the comparison image generated in S2140. At this time, the comparison image database 24 also records information (e.g., examination image ID, supplementary information, such as the examination date) about each examination image from which the comparison image is generated, in addition to the information about the comparison image generated in S2140. This allows the medical information processing apparatus 50 to acquire, from the comparison image database 24, both the comparison image and the information about the examination images from which the comparison image is generated, during the comparison display processing to be performed later.

In S2110 to S2150 described above, the comparison image generation processing according to the present exemplary embodiment is performed.

Next, the comparison display processing performed by the medical information processing apparatus 50 according to the present exemplary embodiment will be described in detail. FIG. 6B is a flowchart illustrating a procedure performed as the comparison display processing by the medical information processing apparatus 50 according to the present exemplary embodiment. The procedure performed as the comparison display processing by the medical information processing apparatus 50 will be described in detail below according to an order specified in the flowchart.

### (S2210) <Acquisition of Comparison Image>

In S2210, the comparison display control unit 39 controls the comparison image acquisition unit 52 and performs processing to acquire a processing target comparison image from the comparison image database 24. At this time, the comparison image acquisition unit 52 acquires a list of comparison images generated by the comparison image generation processing from the comparison image database 24 and displays the acquired list on the display unit 36. Then, a user operation is received via the operation unit 35, and a comparison image of user-interest is acquired from the comparison image list. Specifically, the comparison image ID contained in the supplementary information about the selected comparison image is passed to a subsequent via the storage unit 34.

### (S2220) <Acquisition of Examination Period>

In S2220, the comparison display control unit 39 performs processing to acquire the examination period of the comparison image acquired in S2210 by controlling the examination period acquisition unit 54. The examination period of the comparison image herein refers to a period from an examination date of the older one of the two examination images used in generating the comparison image in the comparison image generation processing, to an examination date of the newer one of the two examination images. The examination images used in generating the comparison image can be acquired from the information recorded as the supplementary information about the comparison image.

### (S2230) <Search for Clinical Practice Information>

In S2230, the comparison display control unit 39 controls the clinical practice information acquisition unit 43 and performs processing to search for and acquire the clinical practice information relevant to the comparison image acquired in S2210, from the clinical practice information database 22. This processing is realized by a system of determining whether there is a relationship based on the examination period acquired in S2220. Details of the processing are similar to those in S1040 in the first exemplary embodiment. Detailed description thereof is omitted here.

### (S2240) <Display>

In S2240, the comparison display control unit 39 controls the display processing unit 45 and performs processing to control the screen display in such a manner that the user can refer to the comparison image acquired in S2210 and the clinical practice information acquired in S2230. Details of the processing are similar to those in S1050 in the first exemplary embodiment. Detailed description thereof is omitted here.

Through the above-described procedure, the user can select a desired comparison image and the medical information processing apparatus 50 according to the present exemplary embodiment searches for the clinical practice information that is relevant to the selected comparison image based on the examination dates of the examination images from which the comparison image is generated. Then, the medical information processing apparatus 50 performs processing to display the extracted clinical practice information in such a manner that the user can refer to the clinical practice information together with the comparison image. In the present exemplary embodiment, the comparison image is generated prior to the comparison display processing for presenting a comparison display to the user, compared to the first exemplary embodiment. Thus, the user does not have to wait for the completion of the comparison image generation processing during the comparison display processing. This produces an advantage that the medical information processing system can be provided that requires the user to devote a less time.

### MODIFIED EXAMPLE 2-1

While the search and acquisition of the clinical practice information that is relevant to the comparison image in the comparison display processing has been described in S2230 as an example in the present exemplary embodiment, the implementation of some embodiments is not limited to that described above. For example, after the comparison image generation processing in S2140, the comparison image generation unit 44 may execute processing similar to that in S2230 on each of the plurality of comparison images generated in S2140 and acquire relevant clinical practice information for each of the plurality of comparison images. Then, in S2150, the relevant clinical practice information may be recorded as supplementary information at the time of recording the comparison image. In this case, in the comparison display processing in S2230, the clinical practice information acquisition unit 43 can acquire the relevant clinical practice information by reading the clinical practice information recorded as the supplementary information about the comparison image. This produces an advantage that the clinical practice information search processing is finished prior to the comparison display processing so that a highly-convenient system can be provided that requires the user to devote a less time. Especially in a case of a clinical practice information search using a complicated condition (e.g., condition that requires advanced recognition processing regarding details of a clinical practice), the search may take a long time. Accordingly, the advantage that the processing is finished in advance is significant.

In the first exemplary embodiment, the example of the medical information processing apparatus configured to receive selection of the first examination image and the second examination image from the user has been described. In the second exemplary embodiment, the example of the medical information processing apparatus configured to receive selection of the comparison image from the user has been described. In a third exemplary embodiment, a medical information processing apparatus configured to receive selection of clinical practice information from the user and acquire a comparison image using a first examination image and a second examination image identified based on the selected clinical practice information will be described.

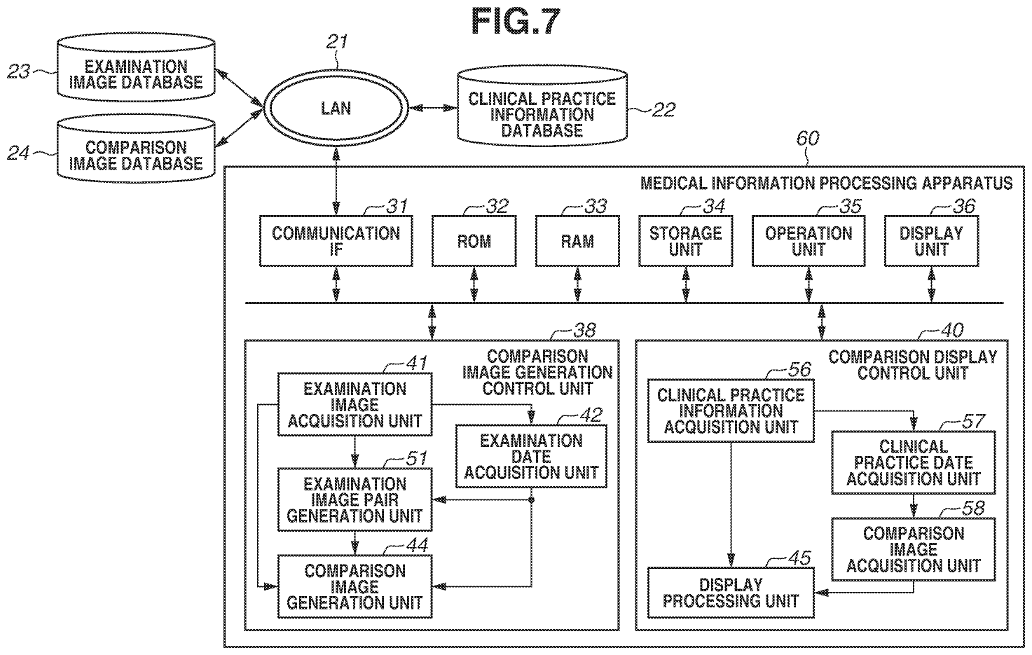

FIG. 7 illustrates an entire configuration of the medical information processing system according to the third exemplary embodiment. The medical information processing system includes a medical information processing apparatus 60, the clinical practice information database 22, the examination image database 23, and the comparison image database 24, all of which are communicably connected to one another via the communication unit. A configuration having a similar function to that described in the first or second exemplary embodiment is given the same reference number, and detailed description thereof is omitted. A difference from the first and second exemplary embodiments will be described below.

The medical information processing apparatus 60 acquires information stored in the clinical practice information database 22, the examination image database 23, or the comparison image database 24 via the LAN 21. A functional configuration of the medical information processing apparatus 60 includes the communication IF 31, the ROM 32, the RAM 33, the storage unit 34, the operation unit 35, the display unit 36, the comparison image generation control unit 38, and a comparison display control unit 40.

The comparison display control unit 40 is realized by, for example, a CPU and comprehensively controls the comparison display performed by the medical information processing apparatus 60. A functional configuration of the comparison display control unit 40 includes a clinical practice information acquisition unit 56, a clinical practice date acquisition unit 57, a comparison image acquisition unit 58, and the display processing unit 45.

The clinical practice information acquisition unit 56 acquires information of interest among the clinical practice information about the patient, from the clinical practice information database 22 via the communication IF 31 and the LAN 21, according to a user operation input via the operation unit 35. Then, the clinical practice information acquisition unit 56 outputs the acquired clinical practice information to the clinical practice date acquisition unit 57 and the display processing unit 45.

The clinical practice date acquisition unit 57 acquires clinical practice date information with respect to the clinical practice information acquired by the clinical practice information acquisition unit 56. Then, the clinical practice date acquisition unit 57 outputs the acquired clinical practice date information to the comparison image acquisition unit 58.

The comparison image acquisition unit 58 performs processing described below based on the clinical practice date information acquired by the clinical practice date acquisition unit 57 and reads a comparison image to be presented to the user from the comparison image database 24 via the communication IF 31 and the LAN 21. Then, the comparison image acquisition unit 58 outputs the read comparison image to the display processing unit 45.

An entire procedure of the processing performed by the medical information processing apparatus 60 according to the present exemplary embodiment will be described in detail below. The medical information processing apparatus 60 according to the present exemplary embodiment performs two types of processing, i.e., the comparison image generation processing and the comparison display processing. In the comparison image generation processing, a plurality of comparison images is generated based on examination images recorded in the examination image database 23. In the comparison display processing, the comparison images generated by the comparison image generation processing and clinical practice information relevant to the generated comparison images are displayed in such a manner that the user can observe and refer to the comparison images, and the clinical practice information relevant to the comparison images. The comparison image generation processing is performed prior to the comparison display processing.

The comparison image generation processing is similar to the processing in S2110 to S2150 described above in the second exemplary embodiment and is performed by the comparison image generation control unit 38. Detailed description thereof is omitted here.

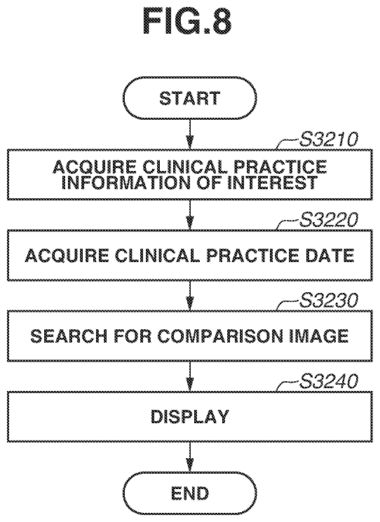

FIG. 8 is a flowchart illustrating the comparison display processing performed by the medical information processing apparatus 60. A procedure of the processing performed as the comparison display processing by the medical information processing apparatus 60 will be described in detail below according to an order specified in the flowchart.

### (S3210) <Acquisition of Clinical Practice Information of Interest>

In S3210, the comparison display control unit 40 controls the clinical practice information acquisition unit 56 and performs processing to acquire clinical practice information of user interest from the clinical practice information database 22. Specifically, the clinical practice information acquisition unit 56 corresponds to one example of a first acquisition unit configured to acquire clinical practice information. In a specific example, a clinical practice information list of all the previous clinical practices regarding the processing target patient is acquired from the clinical practice information database 22 and displayed on the display unit 36, and clinical practice information interesting for the user (clinical practice information of interest) is acquired from among the list via the operation unit 35.

### (S3220) <Acquisition of Clinical Practice Date>

In S3220, the comparison display control unit 40 controls the clinical practice date acquisition unit 57 and performs processing to acquire the clinical practice date contained in the clinical practice information acquired in S3210.

### (S3230) <Search for Comparison Image>

In S3230, the comparison display control unit 40 controls the comparison image acquisition unit 58 and performs processing to acquire, from the comparison image database 24, a comparison image relating to the clinical practice information acquired in S3210. Specifically, the comparison display control unit 40 searches for and acquires a comparison image generated using an examination image of an examination date at or before the clinical practice date and an examination image of an examination date at or after the clinical practice date. More specifically, the comparison image acquisition unit 58 corresponds to one example of a second acquisition unit configured to acquire a comparison image generated using a first examination image acquired in an examination performed before a clinical practice relating to the clinical practice information and a second examination image acquired in an examination performed after the clinical practice. In this way, the comparison image that is relevant to the clinical practice information acquired in S3210 is acquired based on the relationship between the examination date and the clinical practice date.

### (S3240) <Display>

In S3240, the comparison display control unit 40 controls the display processing unit 45 and performs processing to display the clinical practice information acquired in S3210 and the comparison image acquired in S3230 in such a manner that the user can refer to the clinical practice information and the comparison image. The processing performed by the display processing unit 45 in S3240 is similar to that in S2240 in the second exemplary embodiment. Description thereof is omitted here.

By the above-described procedure, the medical information processing apparatus 60 according to the present exemplary embodiment searches for a comparison image in an examination period including a clinical practice date of user-selected clinical practice information, and displays the comparison image in such a manner that the user can refer to the comparison image together with the clinical practice information. This provides a system by which the user can refer to a comparison image that is highly relevant to user-selected clinical practice information by an easy operation.

### MODIFIED EXAMPLE 3-1

The comparison image generation processing performed prior to the comparison display processing has been described as an example in the present exemplary embodiment. The implementation of some embodiments is not limited to that described above. For example, the comparison image generation processing may be performed following the comparison display processing in S3220. In this case, an examination image of an examination date at or before the clinical practice date acquired in S3220 and an examination image of an examination date at or after the acquired clinical practice date are identified based on the acquired clinical practice date. Then, a comparison image is generated using the pair of identified examination images. More specifically, a comparison image is generated using only a pair of examination images in an examination period including the clinical practice date. In this way, the comparison image generation processing does not have to be performed prior to the comparison display processing, and only a smaller number of limited comparison images are generated, so that an advantage is produced that the capacity of the comparison image database 24 is saved and calculation processing is reduced.

### MODIFIED EXAMPLE 3-2

A single piece of clinical practice information in S3210 is acquired as an example in the present exemplary embodiment. However, the implementation of some embodiments is not limited to that described above. For example, a plurality of pieces of clinical practice information may be acquired in S3210, and the clinical practice dates of each piece of clinical practice information may be acquired in S3220. At this time, in S3230, the comparison image database 24 may be searched to acquire a comparison image in an examination period including the clinical practice dates of the plurality of pieces of clinical practice information. The above-described technique provides a system for searching for and observing a comparison image that is relevant to a series of clinical practices in a case where a surgery or medication is performed over a plurality of dates.

While the case of acquiring a comparison image of an examination period including a plurality of clinical practice dates has been described above as an example, a comparison image of an examination period including at least one of the plurality of clinical practice dates may be acquired. Through this processing, a system for searching for and observing a comparison image that is relevant to at least one of the clinical practices performed over a plurality of clinical practice dates can be provided.

### Other Embodiments

Some embodiment(s) can also be realized by a computer of a system or apparatus that reads out and executes computer-executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer-executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer-executable instructions. The computer-executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has described exemplary embodiments, it is to be understood that various embodiments are not limited to the disclosed exemplary embodiments. The scope of the following claims is to be accorded the broadest interpretation so as to encompass all such modifications and equivalent structures and functions.

This application claims priority to Japanese Patent Application No. 2018-201985, which was filed on Oct. 26, 2018 and which is hereby incorporated by reference herein in its entirety.

What is claimed is:

1. An information processing apparatus comprising:
an acquisition unit configured to acquire a comparison image generated using a first examination image and a second examination image which are acquired by imaging a subject at different times/dates from each other;
a search unit configured to search for clinical practice information about a clinical practice performed on the subject during a period defined by a first examination date, at which the first examination image is acquired, and a second examination date, at which the second examination image is acquired; and
a display control unit configured to display the comparison image and the searched clinical practice information on a display unit.

2. The information processing apparatus according to claim 1, wherein the search unit searches the searched clinical practice information for clinical practice information that specifies a detail of a clinical practice that may have caused a change between the first examination image and the second examination image.

3. The information processing apparatus according to claim 2, wherein the detail of the clinical practice that may have caused the change between the first examination image and the second examination image, includes at least one of a medical treatment with a drug and a surgical medical treatment.

4. The information processing apparatus according to claim 1,
wherein the comparison image is associated with info' cation specifying the first examination date and information specifying the second examination date, and
wherein the search unit acquires the first examination date and the second examination date from the comparison image.

5. The information processing apparatus according to claim 1, further comprising a reception unit configured to receive input of the first examination date and the second examination date,
wherein the acquisition unit acquires the comparison image generated using the first examination image identified based on the received input first examination date and the second examination image identified based on the received input second examination date.

6. The information processing apparatus according to claim 1, wherein the comparison image is an image specifying a difference between the first examination image and the second examination image.

7. The information processing apparatus according to claim 1, further comprising a recording unit configured to record the comparison image and the searched clinical practice information in association with each other.

8. An information processing apparatus comprising:
an acquisition unit configured to acquire a comparison image generated using a first examination image and a second examination image which are acquired by imaging a subject at different times/dates from each other, and clinical practice information about a clinical practice performed on the subject;
a display control unit configured to display the comparison image and the clinical practice infoimation on a display unit; and
a determination unit configured to determine whether the clinical practice relating to the clinical practice information is performed during a period defined by a first examination date, at which the first examination image is acquired, and a second examination date, at which the second examination image is acquired,
wherein the display control unit displays the clinical practice information on the display unit in such a manner that the clinical practice information is discriminable from other clinical practice information, based on a result of the determination.

9. The information processing apparatus according to claim 1, wherein the period is a period from the first examination date to the second examination date.

10. The information processing apparatus according to claim 1, wherein the period is a time period from a date prior to the first examination date to the second examination date.

11. An information processing apparatus comprising:
a first acquisition unit configured to acquire clinical practice information;
a second acquisition unit configured to acquire a comparison image generated using a first examination image acquired in an examination performed before a clinical practice relating to the clinical practice information and a second examination image acquired in an examination performed after the clinical practice; and
a display control unit configured to display the comparison image and the clinical practice information on a display unit.

12. An infonnation processing method comprising:
acquiring a comparison image generated using a first examination image and a second examination image which are acquired by imaging a subject at different times/dates from each other;
searching for clinical practice information about a clinical practice performed on the subject during a period defined by a first examination date, at which the first examination image is acquired, and a second examination date, at which the second examination image is acquired; and
performing display control to display the comparison image and the searched clinical practice information on a display unit.

13. An infonnation processing method comprising:
acquiring a comparison image generated using a first examination image and a second examination image which are acquired by imaging a subject at different times/dates from each other and clinical practice information about a clinical practice performed on the subject;
performing display control to display the comparison image and the clinical practice information on a display unit; and
determining whether the clinical practice relating to the clinical practice information has been performed during a period defined by a first examination date, at which the first examination image is acquired, and a second examination date, at which the second examination image is acquired,
wherein the display control is performed to display the clinical practice information on the display unit in such a manner that the clinical practice information is discriminable from other clinical practice information, based on a result of the determination.

14. An information processing method comprising:
acquiring clinical practice information;
acquiring a comparison image generated using a first examination image acquired in an examination performed before a clinical practice relating to the clinical practice information and a second examination image acquired in an examination performed after the clinical practice; and
performing display control to display the comparison image and the clinical practice information on a display unit.

15. A non-transitory computer-readable storage medium that stores a program for causing a computer to execute each unit of the information processing apparatus according to claim 1.
